(19) United States
(12) Patent Application Publication
(10) Pub. No.: US 2003/0207843 A1
(43) Pub. Date: Nov. 6, 2003

(54) Treatment of drug-resistant human immunodeficiency virus infection

(76) Inventors: Karl Y. Hostetler, Del Mar, CA (US); John W. Mellors, Pittsburgh, PA (US)

Correspondence Address: Lisa A Haile, Gray Cary Ware & Freidenrich, Suite 1100, 4365 Excutive Drive, San Diego, CA, 92121-2133, US

(21) Appl. No.: 10/169,432

(22) PCT Filed: Dec. 22, 2000
(86) PCT No.: PCT/US00/35137

(30) Foreign Application Priority Data
Dec. 29, 1999 (US) 60173610
Jan. 4, 2000 (US) 60174425

(51) Int. Cl. A61K 31/66

(52) U.S. Cl. 514/114; 514/125

(57) ABSTRACT

The invention provides methods for treating HIV infection in a subject in need thereof comprising lipid analogs of phosphonoformate-containing pharmaceutically active compounds. Lipid analogs contemplated for use in the practice of the present invention comprise phosphonoformates covalently linked (directly or indirectly through a linker molecule) to a substituted or unsubstituted alkylglycerol, alkylpropanediol, alkylethanediol, or related moiety. In particular, the invention provides methods for treating viral infections caused by viruses which have developed resistance to currently available antiviral agents, as well as methods comprising the use of invention compounds in combination with azidodeoxythymidine to minimize the selection of drug-resistant HIV variants during therapy.

### FIELD OF THE INVENTION

The present invention relates to methods for the treatment of human immunodeficiency virus (HIV) infection. The invention relates particularly to methods for the treatment of viral infections caused by HIV variants which have developed resistance to antiviral agents which inhibit the replication of said viruses.

### BACKGROUND OF THE INVENTION

Antiviral agents have been used successfully to treat a variety of diseases caused by viral infections. These agents effectively treat viral infections by inhibiting replication of the virus, for example, by interfering with the action of viral polymerase replication enzymes. In particular, infections caused by HIV have been effectively treated by agents which interfere with the normal function of viral reverse transcriptase (RT), a replication enzyme common to all retroviruses.

Phosphonoformate (PFA or foscarnet) is a potent antiviral agent due to its ability to inhibit the action of viral polymerases such as, for example, DNA and RNA polymerases and reverse transcriptase (Crumpacker, C. S., 1992 The American Journal of Medicine 92(SA); 3S-7S). As such, it has been demonstrated that foscarnet effectively inhibits the growth of viruses such as herpes simplex virus, influenza, cytomegalovirus, and retroviruses such as HIV-1 (Bacigalup, et. al., 1994 Bone Marrow Transplantation 13: 753-758; Verdonck, et. al., 1993 ibid.11: 177-179; Wagstaff, et. al., 1994 Drugs 48: 199-226).

Phosphonoformate is an analog of pyrophosphate, which is a biproduct of nucleotide polymerization and is composed of the β and γ phosphates cleaved from the incoming nucleotide triphosphate during incorporation into the nascent DNA strand. Without wishing to be bound by theory, PFA is thought to inhibit the replication of retroviruses such as HIV-1, by binding to reverse transcriptase, preventing the binding of the next nucleoside triphosphate, thereby blocking further catalysis. The use of PFA to treat viral infections has been previously described. See, for example, Chrisp, et. al., Drugs, 41: 104-129 (1991); Beadle, et al. Antiviral Chem Chemother 9:33-40 (1997); Beadle, et al., Antiviral Chem Chemother 9:33-40 (1988); Hostetler, et al., Antiviral Res. 31:59-67 (1996); Hostetler, et al, Antiviral Chem Chemother, 11:213-220 (2000); Kini, et al. Anitiviral Res 36:43-53 (1997); Kini, et al. Antiviral Res 36:115-124 (1997); Mellors, et. al., Mol. Pharmacol. 43:11-16 (1992); Nguyen, et. al., Antimicrob. Agents Chemother., 38:2409-2414 (1994).

Although progress has been made in the treatment of AIDS through the use of antiretroviral agents, an unfortunate result of this treatment is that these same antiretroviral agents place HIV-1 under selective pressure to mutate, leading to drug insensitivity and poor treatment outcome (Richman, Scientific American, 1988 279:88). Development of viral resistance to antiretroviral drugs used for treatment of HIV infection is a leading cause of treatment failure and limits options for alternative antiretroviral regimens. Resistance has arisen to all nucleoside inhibitors of HIV reverse transcriptase (NRTI's) including the nucleoside analogs zidovudine (AZT), didanosine (ddI), zalcitabine (ddC), lamivudine (3TC), stavudine (d4T), and abacavir, as well as to non-nucleoside reverse transcriptase inhibitors (NNRTI's) such as nevirapine, delavirdine, and efavirenz (Hirsch, et. al., JAMA 1998 279:1984-1991). Additionally, resistance has developed to HIV-1 protease inhibitors such as saquinavir, indinavir, ritonavir, agenerase, and DMP450.

Accordingly, there is a need in the art for antiviral agents which effectively hinder replication of HIV variants which have developed resistance to inhibition of viral replication by currently available treatment regimens.

### BRIEF DESCRIPTION OF THE INVENTION

The invention provedes methods for treating viral infection in a subject in need thereof comprising administering to said subject an effective amount of one or more lipid analogs of phosphonoformate or thiophosphonoformate. Lipid analogs contemplated for use in the practice of the present invention comprise phosphonoformates covalently linked (directly or indirectly through a linker molecule) to a substituted or unsubstituted alkylglycerol, alkylpropanediol, alkylethanediol, or related moiety. In particular, the invention provides methods for treating viral infections caused by viruses which have developed resistance to currently available antiviral agents.

In one aspect of the invention, there are provided methods for treating viral infections caused by retroviruses. In a particular aspect of the invention, there are provided methods for treating viral infections caused by retroviruses which have developed resistance to antivirals, such as, for example, reverse transcriptase inhibitors, and the like.

In another aspect of the invention, there are provided methods for treating viral infections comprising administering to a subject in need thereof an effective amount of a lipid analog of a phosphonoformate in combination with azidothymidine (AZT), an HIV reverse transcriptase inhibitor.

### BRIEF DESCRIPTION OF THE FIGURES

FIG. 1 illustrates exemplary structures of phosphonoformate lipid analogs contemplated for use in the practice of the present invention.

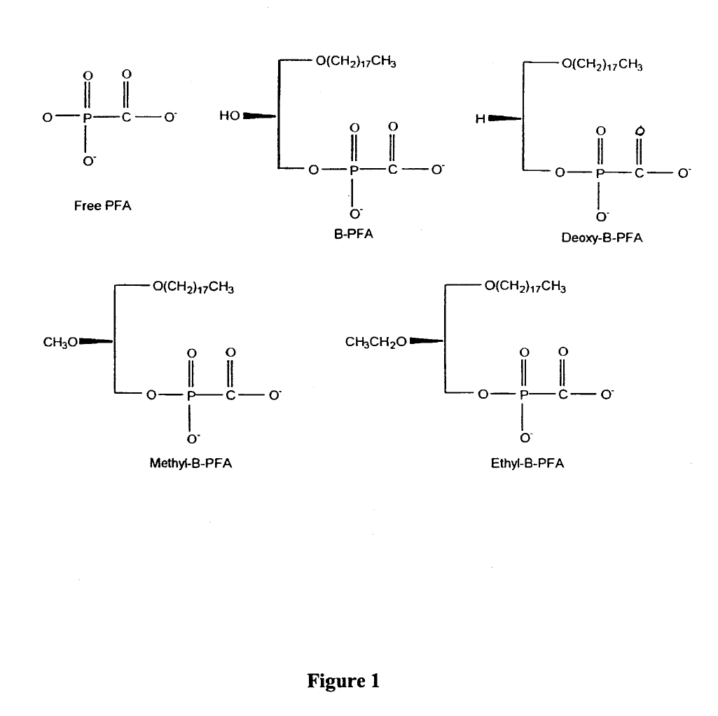

### DETAILED DESCRIPTION OF THE INVENTION

In accordance with the present invention, there are provided methods for treating viral infections in a subject in need thereof. Indications appropriate to such therapeutic intervention include susceptible viruses such as human immunodeficiency virus (HIV) in which the reverse transcriptase of said virus has become resistant to various reverse transcriptase inhibitors, such as, for example, nucleoside reverse transcriptase inhibitors (NRTI's), non-nucleoside reverse transcriptase inhibitors (NNRTI's), and the like.

The methods of the present invention comprise administering to the subject an effective amount of a lipid analog of a phosphonoformate or thiophosphonoformate, wherein the lipid analog has the following structure:

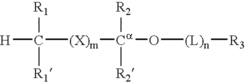

wherein:

R₁ and R₁′ are independently —H, optionally substituted —O(C₁-C₂₄)alkyl, —O(C₁-C₂₄)alkenyl, —S(C₁-C₂₄)alkyl, —S(C₁-C₂₄)alkenyl, —O(C₁-C₂₄)acyl, —S(C₁-C₂₄)acyl, wherein at least one of R₁ and R₁′ are not —H, and wherein said alkenyl has 1 to about 6 double bonds, and said acyl optionally has 1 to about 6 double bonds;

R₂ and R₂′ are independently —H, optionally substituted —O(C₁-C₇)alkyl, —O(C₁-C₇)alkenyl, —S(C₁-C₇)alkyl, —S(C₁-C₇)alkenyl, —O(C₁-C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;

R₃ is a phosphonoformate which is linked, either through its carboxyl group or its phosphonate group, to a functional group on optional linker L or to an available oxygen on C^{α}, wherein when R₃ is linked through its phosphonate group, the carboxylate group of said phosphonoformate has the following structure:

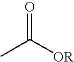

wherein:

R_{y} is —H or alkyl, or

Na⁺, K⁺, NH₄⁺, or any other physiologically acceptable cation,

X, when present, is:

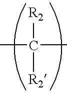

L, when present, is a bifunctional linking molecule of the formula -J-(CR₂)ₜ-G-, wherein t is an integer from 1 to 24, J and G are independently —O—, —S—, —C(O)O—, or —NH—, and R is —H, alkyl, or alkenyl;

m is an integer from 0 to 6; and

n is 0 or 1.

As used herein, the term “alkyl” refers to a monovalent straight or branched chain or cyclic radical of from one to twenty-four carbon atoms, including methyl, ethyl, n-propyl, isopropyl, n-butyl, isobutyl, tert-butyl, n-hexyl, and the like.

As used herein, “substituted alkyl” comprises alkyl groups further bearing one or more substituents selected from hydroxy, alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As used herein, “alkenyl” refers to straight or branched chain hydrocarbyl groups having one or more carbon-carbon double bonds, and having in the range of about 2 up to 24 carbon atoms, and “substituted alkenyl” refers to alkenyl groups further bearing one or more substituents as set forth above.

Phosphonoformates contemplated for use in the practice of the present invention include, for example, the batyl alcohol adduct, 1-O-octadecylglycero-3-PFA (B-PFA), 1-O-octadecyl-2-O-methylglycero-3-PFA (MB-PFA), 1-O-octadecyl-2-O-ethyl-glycero-3-PFA (EB-PFA), and the compounds described in U.S. Pat. Nos. 5,194,654, 5,411,947 5,463,092, 5,696,277, 5,744,461, and 6,002,029; and in U.S. application Ser. Nos. 08/487,081 and 08/986,881, which are hereby incorporated by reference in their entirety.

Preferred lipids comprise substituted glycerol, propanediol, butanediol, and ethanediol groups. Particularly preferred lipids comprise glycerol moieties.

Preferred lipid analogs of the invention have the following structure:

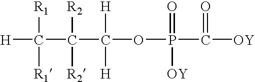

wherein:

R₁ is O(C₁₈alkyl), R₁′ and R₂′ are each —H, and R₂ is —OH, OMethyl, or OEthyl, wherein the resulting analogs are referred to as B-PFA, MB-PFA, or EB-PFA, respectively, and Y is a physiologically acceptable cation such as, for example, Na⁺, K⁺, NH₄⁺, and the like.

The methods of the present invention are particularly effective for treatment of infections caused by retroviruses such as, for example, HIV-1, and the like.

In preferred embodiments, the methods of the present invention are useful in treating infections caused by a mammalian immunodeficiency virus, such as, for example, HIV-1. The methods of the present invention are particularly effective for treatment of infections caused by variants of HIV-1 which have become resistant to inhibitor(s) of reverse transcriptases (RT). RT inhibitors include, for example, the nucleoside analogs zidovudine (AZT), didanosine (ddI), zalcitabine (ddC), lamivudine (3TC), stavudine (d4T), emirivine (FTC), abacavir, and the like, as well as non-nucleoside analogs such as, for example, nevirapine, delavirdine, efavirenz, and the like.

The methods of the present invention are also useful for treatment of infections caused by HIV-1 variants which have developed resistance to protease inhibitors, such as, for example, saquinavir, indinavir, ritonavir, agenerase, DMP450, and the like.

Further provided by the present invention are methods for the prevention or treatment of a pathological condition caused by a viral infection in a subject in need thereof, said method comprising administering to said subject an effective amount of a lipid analog of a phosphonoformate.

Still further provided by the present invention are methods for treating a viral infection in a mammal, said method comprising administering to a subject in need thereof an effective amount of a lipid analog of a phosphonoformate in combination with an HIV reverse transcriptase inhibitor, such as, for example, zidovudine (AZT, azidodeoxythymidine), which selects for mutations which sensitize the HIV variant to the PFA compounds of the invention. Conversely, the compounds of the invention select for HIV RT mutations which reverse or reduce AZT resistance.

The lipid analogs described herein have been shown to be orally bioavailable (Beadle, et. al., Antiviral Chem. Chemo., 1998 9:3340). AIDS patients infected with drug-resistant strains of HIV can be treated preferably by oral administration of the lipid analogs described herein. Thus, compounds of the invention can be administered in a varity of ways, e.g., in the form of tablets, capsules, solutions, emulsions or suspensions, inhaled liquid or solid particles, microencapsulated particles, as a spray, through the skin by an appliance such as a transdermal patch, rectally, for example, in the form of suppositories, and the like. The lipophilic derivatives of the invention are particularly well suited for transdermal absorption administration and delivery systems and may also be used in toothpaste. Administration can also take place parenterally in the form of injectable solutions, for intravenous, subcutaneous, intraperitoneal, cisternal administration, and the like.

The pharmaceutical carrier or diluent employed in the practice of the present invention may be a conventional solid or liquid carrier. Examples of solid carriers are lactose, sucrose, talc, gelatin, agar, pectin, acacia, magnesium stearate, stearic acid, or lower alkyl ethers of cellulose. Examples of liquid carriers are syrup, peanut oil, olive oil, phospholipids, fatty acids, fatty acid amines, polyoxyethylene or water. The carrier or diluent may include any sustained release material known in the art, such as glyceryl monostearate or distearate, alone or mixed with a wax.

If a solid carrier is used for oral administration, the preparation may be tabletted or placed in a hard gelatin capsule in powder or pellet form. The amount of solid carrier will vary widely, but will usually be from about 25 mg to about 1 gm. If a liquid carrier is used, the preparation may be in the form of a syrup, emulsion, soft gelatin capsule, sterile injectable liquid such as an aqueous or non-aqueous liquid suspension or solution, and the like.

Tablets are prepared by mixing the active ingredient (that is, one or more compounds of the invention), with pharmaceutically inert, inorganic or organic carrier, diluents, and/or excipients. Examples of such excipients which can be used for the preparation of tablets include lactose, maize starch or derivatives thereof, talc, stearic acid or salts thereof. Examples of suitable excipients for gelatin capsules are vegetable oils, waxes, fats, semisolid, and liquid polyols. The lipid analogs can also be made in microencapsulated form.

For nasal administration, the preparation may contain a compound of the invention dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

The present invention embraces the use of pharmaceutical compositions for containing pharmaceutically acceptable sterile aqueous or non-aqueous liquids, dispersions, suspensions or emulsions as well as sterile powders for reconstitution into sterile injectable solutions or dispersions just prior to use parenteral injection. Pharmaceutical formulations containing compounds of this invention can be prepared by conventional techniques, e.g., as described in Remington's Pharmaceutical Sciences, 1985.

Suitable excipients for the preparation of solutions and syrups are water, polyols, sucrose, invert sugar, glucose, liposomes, and the like. Suitable excipients for the preparation of injectable solutions are water, alcohols, polyols, glycerol, vegetable oils, and the like.

The pharmaceutical products can additionally contain any of a variety of added components, such as, for example, preservatives, solubilizers, stabilizers, wetting agents, emulsifiers, sweeteners, colorants, flavorings, buffers, coating agents, antioxidants, diluents, and the like.

Optionally, the present invention also embraces pharmaceutical compositions containing a lipid analog as described herein in combination with one or more compounds exhibiting a different activity, for example, an antibiotic or other pharmacologically active material. Such combinations and use thereof are within the scope of the invention.

The term “effective amount” as applied to the lipid analogs of the present invention is an amount that will prevent or reverse the disorders associated with viral infections noted above. The “effective amount” is determined with reference to the recommended dosages of the antiviral parent compound. The selected dosage will vary depending on the activity of the selected compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient will depend on a variety of factors, including the body weight, general health, diet, time, and route of administration and combination with other drugs, and the severity of the disease being treated.

Generally, the compounds of the present invention are dispensed in unit dosage form comprising 1% to 100% of active ingredient. The range of therapeutic dosage is from about 0.01 to about 1,000 mg/kg/day with from about 0.10 mg/kg/day to 100 mg/kg/day being preferred, when administered to patients, e.g., humans, as a drug. Actual dosage levels of active ingredients in the pharmaceutical compositions of this invention may be varied so as to administer an amount of the active compound(s) that is effective to achieve the desired therapeutic response for a particular patient.

The invention will now be described in greater detail by reference to the following non-limiting examples.

### EXAMPLES

Chemicals. The following ccmpounds were prepared as 5 mM liposomal preparations as previously reported (see Hostetler, et. al., Antiviral Chemistry and Chemotherapy 11: 213-220, and Kini, et. al., Antiviral Research 36: 43-53.): 1-O-octadecyl-sn-glycero-3-PFA (B-PFA), 1-O-octadecyl-propanediol-3-PFA (DB-PFA), 1-O-octadecyl-2-O-methyl-sn-glycero-3-PFA (MB-PFA) and 1-O-octadecyl-2-O-ethyl-sn-glycero-3-PFA (EB-PFA). The compounds were stored at 4° C. and warmed to 37° C. immediately before use. 3′-Azido-3′-deoxythymidine (AZT) and phosphonoformate (PFA) were purchased from Sigma Chemical Company, St. Louis, Mo. AZT and PFA were prepared as 10 or 30 mM stock solutions in DMSO and sterile water, respectively, and stored at −20° C. Immediately before use, the compounds were warmed to 37° C. and diluted to the desired concentrations in RPMI 1640 medium.

Cells. MT-2 cells (AIDS Research and Reference Reagent Program, National Institute of Allergy and Infectious Diseases, National Institutes of Health) were maintained in RPMI 1640 supplemented with 10% fetal bovine serum (FBS; JRH Biosciences, Lenexa, Kans.), 10 mM HEPES buffer, 50 IU/ml penicillin and 50 μg/ml streptomycin.

Viruses. Stock virus was prepared by electroporating (BIO-RAD Gene Pulser®, Hercules, Calif.) MT-2 cells (1.3×10⁷ cells) with 5-10 μg of plasmid DNA encoding a proviral clone of HIV-1_{LAI} (see Nguyen, et. al., Antimicrob. Agents Chemother. 38:2409-2414, and Peden, et. al., Virology 185: 661-672.). At peak cytopathic effect (generally 5-7 days post-transfection), culture supernatants were harvested and stored at −80° C. Viral infectivity titers were determined in threefold endpoint dilution assays conducted in MT-2 cells (six wells per dilution). The 50% tissue culture infective dose (TCID₅₀) was calculated using the Reed and Muench equation (see Reed, et. al., Am. J. Hyg. 27:493-496).

Antiviral Susceptibility Assays. The antiviral activity of each compound was determined by inoculating MT-2 cells with HIV-1_{LAI} at a multiplicity of infection (MOI) of 0.01 TCID₅₀/cell, followed by incubation in the presence of three-fold serial drug dilutions (3 wells per dilution) (see Mellors, et. al., Antimicrob. Agents Chemother. 39:1087-1092). Five or seven days after infection, culture supernatants were harvested, lysed with 0.5% triton-X 100 and assayed for p24 antigen concentration using a commercial ELISA assay (DuPont, NEN Products, Wilmington, Del.). The antiviral activity of the compounds is expressed as the IC₅₀, which is the concentration required to inhibit 50% of p24 antigen production. The fold-resistance of a test virus is calculated by dividing the IC₅₀ of the test virus by the IC₅₀ of the HIV-1_{LAI} control virus.

Selection of Resistant Viruses. Selections were initiated by inoculating 1.0×10⁶ MT-2 cells at an MOI of 0.1 with plasmid derived HIV-1_{LAI} which had been passaged as cell-free virus 10 times in MT-2 cells in the absence of compound (see Bazmi, et. al., Antimicrobial Agents and Chemotherapy 44:1783-1788). Cells were pretreated with drug for two hours prior to inoculation with virus. For each selection, the starting concentration was the IC₅₀ of the compound, and the selective pressure (i.e. drug concentration) was doubled every three passages. Viral cytopathic effects (CPE) were monitored daily. At 2+CPE (≧2 syncitia per 100× field) cell free virus supernatant was harvested and used to initiate a new cycle of infection in fresh MT-2 cells. The passaged virus was monitored regularly for a reduction in susceptibility to the compounds by determining the EC₅₀ relative to unpassaged HIV-1_{LAI} (Mellors, et. al., Antimicrob. Agents Chemother. 39:1087-1092).

Genetic Analyses. Virions were pelleted from culture supernatants by centrifugation at 25,000×g for 1 hour. Total RNA was extracted from the virus pellet using TRIZOL® Reagent (Gibco BRL, Grand Island, N.Y.) and resuspended in diethyl pyrocarbonate-treated sterile water. After cDNA synthesis, the full-length coding region of RT was amplified using PCR (see Bazmi, et. al., Antimicrobial Agents and Chemotherapy 44:1783-1788). The bulk PCR products were then purified using a commercially available kit (Wizard® PCR Purification System, Promega, Madison, Wis.) and sequenced using an automated sequencer (PE Biosciences, San Francisco, Calif.).

Generation of Mutant Recombinant HIV-1. HIV-1 containing the desired mutations were generated by oligonucleotide directed mutagenesis (Altered Sites II, Promega) as previously described (Mellors, et. al., Molecular Pharm. 41:446-451). After mutagenesis, mutant RT was subcloned into the pxxHIV-1_{LAI} cloning vector using the silent XmaI and XbaI restriction sites located at the 5′ and 3′ ends of RT. Clones were DNA sequenced to verify the presence of the desired mutation(s) and electroporated into MT-2 cells as described above to generate infectious mutant recombinant HIV-1.

Generation of recombinant HIV-1 was performed as described in the cited references to Mellors, et. al. and Nguyen, et. al, which are hereby incorporated by reference.

Mutant RT was generated via oligonucleotide-directed mutagenesis and ligated into the pXXHIV-1_{LAI} cloning vector. Cloning was facilitated by the presence of two silent restriction sites at the 5′ and 3′ ends (Xma1 and Xba1, respectively) of pXXHIV-1_{LAI} RT. Infectious mutant recombinant HIV-1 was generated by electroporating MT-2 cells with mutated pXXHIV-1_{LAI} clones. The presence of the desired mutations was verified by DNA sequencing.

The multi-drug resistant HIV-1 strains, 11163p1 and 11588p1, are human clinical isolates having the RT mutations indicated. HIV-1 strains treated with the lipid analogs of the present invention include wild type, e.g., xxLAI, and the like, single mutation, e.g., xxLAI MI 84V (resistant to 3TC), xxLAI L74V (resistant to ddI and ddC), xxLAI K65R (resistant to ddI, ddC, DAPD, DXG, tenofovir, and adefovir), xxLAI T215Y (resistant to AZT), and the like, double mutations, e.g., xxLAI M41 L/T215Y (resistant to AZT), xxLAI MI 84V/T215Y (resistant to 3TC and AZT), and the like, and multiple mutations, e.g., xxLAI 4XAZT ((D67N, K70R, T215Y, K219Q) resistant to AZT)), xxLAI 4XAZT/M184V ((D67N, K70R, M 184V, T215Y, K219Q) resistant to AZT and 3TC), xxLAI M5456-12 (AZT+NNRTI resistant: D67N, K70R, K103N, T215Y), xxLAI G2-3g (AZT/3TC co-resistant: M41L, D67N, M184V, H208Y, L210W, R211K, L214F, T215Y, 1293V, E297A), 11163 (multinucleoside resistant virsus: M41L, A62V, V751, F77L, K103N, F116Y, Q151M, Y181C, M184V), 11588 (multinucleoside resistant virsus: A62V, K65R, K70R, V751, F77L, F116Y, Q151M, M184V, K219Q), and the like.

The in vitro selectivity indexes for PFA and lipid analogs of PFA were determined in MT-2 cells and are as follows:

| Compound | Selectivity Index |
|---|---|
| PFA (foscarnet) | 137 |
| B-PFA | 781 |
| MB-PFA | 953 |
| EB-PFA | 1905 |

Note that the selectivity index ((50% cytotoxic dose/50% effective dose)×100) is much greater for the lipid analogs of PFA than for PFA itself. Since the lipid analogs have a much greater oral absorption than PFA, they may be given orally for controlling replication of drug-resistant HIV either alone or in combination with one or more antiviral agents. AZT is especially preferred in combination with the lipid analogs of PFA.

### Example 1

Cultures of MT-2 cells infected with either wild type (xxLAI) or with the drug-resistant strains of HIV-1 listed above were treated with lipid analogs of PFA (B-PFA, MB-PFA, or EB-PFA). The μM concentration required to produce 50% inhibition of replication of the drug-resistant strain of HIV (EC₅₀) was determined as shown in Table 1. Drug effects were measured by assaying p24 (a viral protein) in the culture medium.

TABLE 1
Antiviral Activity of B-PFA, MB-PFA, and EB-PFA
Against a Panel of Drug-Resistant Strains of HIV-1

EC₅₀, μM

| Virus | PFA | B-PFA | MB-PFA | EB-PFA |
|---|---|---|---|---|
| HIV-1_{LAI}(control) | 18.3 | 1.9 | 0.35 | 0.22 |
| K65R | 57.3 | 17.7 | 1.24 | 3.22 |
| L74V | 37.4 | 4.25 | 0.52 | 1.34 |
| M184V | 15.7 | 2.29 | 0.48 | 0.58 |
| T215Y | nd | 0.84 | 0.27 | 0.14 |
| M41L/T215Y | 16.2 | 1.25 | 0.13 | 0.41 |
| M184V/T215Y | nd | 1.49 | 0.54 | 0.48 |
| 4xAZT | 8.82 | 1.25 | 0.46 | 0.33 |
| AZT/K103N | 18.6 | 2.72 | 0.50 | 0.80 |
| 4xAZT/M184V | 15.7 | 2.29 | 0.48 | 0.58 |
| G2-3g | 7.6 | 1.47 | 0.26 | 0.32 |
| *The p24 reduction assays were carried out inMT-2 cells infected with an MOI of 0.01. nd = not determined. Virus mutation abbreviations: single or double mutations are given with the standard amino acid letter code followed by the position of HIV RT at which the mutation occurs, followed by the letter code of the amino acid substitution; for multiple virus strains having multiple mutations, 4xAZT = D67N, K70R, T215Y, K219Q; G2-3g = M41L, D67N, M184V, H208Y, L210W, | | | | |
| # R211K, L214F, T214Y, I293V, E297A. | | | | |

When tested against a panel of drug resistant HIV-1 viruses, compounds of the invention, B-PFA, MB-PFA, and EB-PFA, all retained antiviral EC₅₀ values substantially lower than that of foscarnet (PFA). With the exception of K65R, ODG-PFA had EC₅₀ values ranging from 0.84 to 4.25 μM versus 7.6 to 37.4 μM in strains of HIV having single, double, and multiple mutations. MB-PFA and EB-PFA were highly active in drug-resistant HIV with EC₅₀ values generally in the <1 μM range. In K65R, a rare ddI resistant mutant, low level resistance versus wild type was noted (3.5 to 14.6 fold). Note that most drug-resistant HIV strains (Table 1) were studied at a multiplicity of infection (MOI) of 0.01.

### Example 2

Multidrug resistant strains were tested at a 5 times greater MOI of 0.05, the results are shown in Table 2.

TABLE 2
Antiviral Activity of B-PFA, MB-PFA, and
EB-PFA Against MultiDrug-Resistant Strains of HIV-1

EC₅₀, μM

| Virus | PFA | B-PFA | MB-PFA | EB-PFA |
|---|---|---|---|---|
| HIV-1_{LAI}(control) | 20.1 | 3.01 | 1.04 | 0.76 |
| 11163p1 | 24.4 | 3.48 | 1.95 | 1.23 |
| 11588p1 | 23.5 | 9.31 | 1.16 | 0.73 |

The p24 reduction assays were carried out in MT-2 cells infected with an MOI of 0.05. Abbreviations as in Table 1. Mutations of RT present in multidnig-resistant HIV-1: 11163p1 = M41L, A62V, V751, F77L, K103N, F116Y, Q151M, Y181C, M184V; 11588p1 = A62V, K65R, K70R, V751, F77L, F116Y, Q151M, M184V, K219Q.

As shown above, MB-PFA and EB-PFA were highly active even in multidrug resistant strains of HIV-1, the clinical isolates, 11163p1 and 11588p1, even at 5 times higher multiplicity of infection, with EC₅₀ values in the 0.73 to 1.95 μM range.

The activity of three invention compounds against a panel of NRTI-resistant HIV-1 variants was also evaluated (see Examples 3-5). The NRTI-resistant panel consisted of HIV-1_{LAI} derived recombinant viruses containing mutations conferring resistance to numerous NRTIs and included several variants resistant to multiple NRTIs (Tables 3-5).

### Example 3

TABLE 3
Susceptibility of NRTI-Resistant HIV-1 to PFA Invention Compounds

| HIV-1 | EC₅₀^{a,b} (μM) (Fold-resistance)^{c} | | | |
|---|---|---|---|---|
| Variant^{d} | B-PFA | MB-PFA | EB-PFA | Free PFA |
| Wild type | 1.79 ± 0.81 | 0.50 ± 0.48 | 0.65 ± 0.36 | 17.66 ± 10.12 |
| K65R | 14.68 ± 6.61 | 1.66 ± 0.72 | 2.91 ± 1.30 | 67.0 ± 22.79 |
| | (8.2) | (3.3) | (4.5) | (3.8) |
| L74V | 4.45 ± 2.26 | 0.88 ± 0.63 | 1.21 ± 0.85 | 32.7 ± 9.00 |
| | (2.5) | (1.8) | (1.9) | (1.9) |
| M184V | 1.93 ± 0.54 | 1.51 ± 0.45 | 1.36 ± 0.93 | 33.4 ± 18.7 |
| | (0.7) | (1.7) | (0.7) | (1.7) |

^{a}EC₅₀ values determined measuring inhibition of p24 antigen production in MT-2 cells.
^{b}Mean ± standard deviation from at least three independent experiments.
^{c}Fold resistance relative to wild type virus
^{d}HIV-1_{LAI} encoding the indicated resistance mutations

Of the viruses tested, only those containing K65R (ddI/DXG resistant) demonstrated significant resistance to the invention compounds as well as unmodified (free) PFA, with fold-resistance values ranging from 3.3-8.2 (EC₅₀'s of 1.66-14.68 μM). 3TC and ddI/ddC resistant viruses (containing M 184V and L74V resistance mutations, respectively) were sensitive to both the PFA invention compounds and unmodified PFA (fold-resistance<3.0) (Table 3).

### Example 4

Invention compounds were also evaluated against a panel of three multinucleoside resistant (MNR) viruses, as shown in Table 4.

TABLE 4
Susceptibility of Multinucleoside
Resistant HIV-1 to PFA Invention Compounds

| HIV-1 | EC₅₀^{a,b} (μM) (Fold-resistance)^{c} | | | |
|---|---|---|---|---|
| Variant | B-PFA | MB-PFA | EB-PFA | Free PFA |
| HIV-1_{LAI}^{d} | 1.8 ± 0.81 | 0.5 ± 0.48 | 0.7 ± 0.36 | 17.7 ± 10.12 |
| NL4-3^{d} | 2.6 ± 1.5 | 2.3 ± 1.9 | 0.2 ± 0.2 | 15.1 ± 6.1 |
| 11163p3^{e} | 1.1 ± 0.53 | 0.4 ± 0.28 | 0.5 ± 0.06 | 15.5 ± 5.04 |
| | (0.6) | (0.9) | (0.8) | (0.9) |
| K^{f} | 4.2 | 2.0 ± 0.6 | 0.9 ± 0.7 | 21.4 ± 15.6 |
| | (1.6) | (0.87) | (4.5) | (0.7) |
| 9GC^{g} | 4.1 ± 1.2 | 2.3 ± 0.5 | 0.2 ± 0.3 | 13.2 ± 12.8 |
| | (1.6) | (0) | (0) | (1.1) |

^{a}EC₅₀ values determined measuring inhibition of p24 antigen production in MT-2 cells.
^{b} Mean ± standard deviation from two or three independent experiments.
^{c}Fold resistance relative to wild type virus
^{d}Wild type virus
^{e}A multinucleoside resistant clinical isolate with mutations A62V, V75I, F77L, K103N, F116Y, Q151M, Y181C, M184V.
^{f}A multinucleoside resistant recombinant virus with mutations V75I, F77L, F116Y, Q151M.
^{g}A multinucleoside resistant recombinant virus with mutations D67E, S68T, T69S[SA insert], T215Y

The MNR panel consisted of a virus containing the mutations V75I, F77L, F116Y and Q15, a virus containing the T69S[SA insert], and a clinical isolate carrying the classic MNR genotype (62V/751/77L/116Y/151M). Each of the PFA invention compounds retained potency against these viruses with fold resistance values of <2. The sole exception to this was the virus containing 75I/77L/116Y/151M which showed 4.5-fold resistance to EB-PFA.

### Example 5

AZT resistant viruses consisted of HIV-1_{ILA}, derived recombinants with double (M41L/T215Y) and quadruple mutations (D67N, K70R, T215Y, K219Q) (Table 5). These AZT resistant viruses were susceptible to invention compounds and unmodified PFA.

TABLE 5
Susceptibility of AZT Resistant HIV-1 to PFA Invention Compounds

| HIV-1 | EC₅₀^{a,b} (μM) (Fold-resistance)^{c} | | | |
|---|---|---|---|---|
| Variant^{d} | B-PFA | MB-PFA | EB-PFA | Free PFA |
| Wild type | 1.79 ± 0.81 | 0.50 ± 0.48 | 0.65 ± 0.36 | 17.66 ± 10.12 |
| M41L/ | 0.94 ± 1.08 | 0.18 ± 0.11 | 0.30 ± 0.35 | 13.86 ± 4.14 |
| T215Y | (0.5) | (0.4) | (0.5) | (0.8) |
| 4XAZT^{e} | 1.47 ± 0.63 | 0.58 ± 0.22 | 0.34 ± 0.24 | 12.7 ± 8.07 |
| | (0.8) | (1.2) | (0.5) | (0.7) |
| 4XAZT/ | 2.29 ± 1.59 | 0.59 ± 0.36 | 0.56 ± 0.08 | 22.74 ± 15.81 |
| M184V | (1.3) | (1.2) | (0.9) | (1.3) |
| 4XAZT/ | 3.87 ± 2.36 | 0.60 ± 0.38 | 0.88 ± 0.14 | 19.22 ± 8.85 |
| K103N | (2.2) | (1.2) | (1.4) | (1.1) |
| G2-3g^{f} | 1.67 ± 0.95 | 0.40 ± 0.26 | 0.30 ± 0.04 | 8.85 ± 3.49 |
| | (0.9) | (0.8) | (0.5) | (0.5) |

^{a}EC₅₀ values determined measuring inhibition of p24 antigen production in MT-2 cells.
^{b}Mean ± standard deviation from at least three independent experiments.
^{c}Fold resistance relative to wild type virus
^{d}HIV-1_{LAI} encoding the indicated resistance mutations
^{e}4XAZT = D67N, K70R, T215Y, K219Q
^{f}A molecularly cloned isolate co-resistant to AZT and 3TC; M41L, D67N, M184V, H208Y, L210W, R211K, L214F, T215Y, I293V, E297A.

Virus containing the M41L and T215Y mutations consistently showed increased susceptibility to each of the invention compounds compared with wild-type virus: fold changes in EC₅₀'s ranged from 0.4 to 0.53 (EC₅₀ values from 0.18-0.94 μM). Virus containing both the quadruple AZT resistance mutations as well the 3TC resistance mutation M184V (HIV_{4XAZT/M184V}) or the nonnucleoside reverse transcriptase inhbitor (NNRTI) mutation K103N (HIV_{4XAZ/K103N}) also showed sensitivity to the invention compounds (fold-resistance <3.0). Additionally, a molecularly cloned clinical isolate co-resistant to AZT and 3TC (G2-3g) was sensitive to each of the compounds with fold-resistance values <1.0 (EC₅₀ values of 0.30-1.67 μM).

### Example 6

Virus resistant to invention compounds were selected in vitro by serial passage of HIV-1_{LAI} in MT-2 cells in the presence of escalating concentrations of invention compounds, as shown in Table 6.

TABLE 6
Mutations and Altered Susceptibility of
Invention Compound-Resistant HIV-1 Selected In Vitro

| | Virus | | Fold | | |
|---|---|---|---|---|---|
| Compound | Passage | EC₅₀^{a} | resist.^{b} | Codon Δ^{c} | AA Δ^{c} |
| DB-PFA | 0 | 0.83 | — | | |
| | 18 | 8.97 | 10.8 | TCA → ACA | S117T |
| | | | | CTT → TTT | L214F |
| MB-PFA | 0 | 0.07 | — | | |
| | 15 | 2.16 | 30.9 | GTA → TTA | V75L |
| | | | | ATG → ATA | M164I |
| | | | | CTT → TTT | L214F |
| EB-PFA | 0 | 0.09 | — | | |
| | 15 | 3.70 | 41.1 | TGG → GGG | W88G |
| | | | | CTT → TTT | L214F |
| PFA | 0 | 5.27 | — | | |
| | 17 | 122.3 | 23.2 | TGG → GGG | W88G |
| | 15 | 122.2 | 23.2 | TCA → ACA | S117T |

^{a}EC₅₀ values determined measuring inhibition of p24 antigen production in MT-2 cells.
^{b}Fold resistance relative to baseline virus (HIV-1_{LAI} passage 0)
^{c}Change relative to baseline virus (HIV-1_{LAI} passage 0)

Virus exhibiting 27-fold resistance to MB-PFA was isolated after 15 rounds of cell free virus passage. DNA sequencing of the RT gene (amino acid (AA) 1 to 350) from MB-PFA resistant virus identified three mutations: V75L, M164I and L214F. Recombinant viruses encoding the V75L, M164I and L214F mutations were constructed and tested for susceptibility to MB-PFA. Virus containing both M164I and L214F exhibited 9.3-fold resistance (EC₅₀=8.1 μM). The V75L mutation alone or in combination with L214F or M164I did not cause significant (<3-fold) resistance to MB-PFA. Selection of MB-PFA resistant virus was repeated; after 15 passages, the selected virus exhibited 31-fold MB-PFA resistance and encoded the M164I and L214 mutations but not the V75L mutation.

Virus exhibiting 10.8-fold resistance to DB-PFA was isolated after 18 rounds of cell free passage. DNA sequencing identified two mutations in RT: S117T and L214F. Recombinant virus encoding S117T demonstrated 10.0-fold resistance to DB-PFA (EC₅₀=9.0 μM). Addition of the L214F mutation to the virus encoding S117T did not increase the level of DB-PFA resistance (9.7-fold).

Virus exhibiting 41-fold resistance to EB-PFA and having the W88G and L214F mutations was isolated after 15 rounds of cell free passage. Recombinant virus encoding the W88G mutation showed 9.4-fold resistance, which was increased to 15.5-fold resistance (EC₅₀=9.1 μM) when the L214F mutation was added. EB-PFA resistant virus was selected a second time after 15 passages. This virus also contained the mutations W88G and L214F.

As controls for the invention compound selections, HIV-1_{LAI} was passaged in the presence and absence of unmodified PFA. Virus demonstrating 23-fold resistance to PFA was selected after 15 and 17 cycles of cell free passage in two independent selections. DNA sequence analysis of RT from these PFA resistant viruses identified single mutations in RT: W88G (first selection) and S117T (second selection). Recombinant viruses having the W88G and S117T mutations showed 6.2 and 4.7-fold resistance to PFA, respectively. None of the mutations selected by the invention compounds or free PFA were detected in control viruses passaged in the absence of drug.

### Example 7

Invention compounds were also evaluated against a panel of HIV-1LAI derived recombinants resistant to PFA and the PFA invention compounds, as shown in Table 7.

TABLE 7
Invention Compound Susceptibility
of PFA and PFA/AZT Resistant HIV-1

| | EC₅₀^{a,b} (μM) (Fold-resistance)^{c} | | | |
|---|---|---|---|---|
| HIV-1 Variant^{d} | B-PFA | MB-PFA | EB-PFA | Free PFA |
| Wild type | 1.74 ± | 0.77 ± | 0.90 ± | 13.06 ± |
| | 0.79 | 0.33 | 0.73 | 5.59 |
| W88G | >30 | 5.88 ± | 6.8 ± | 152.24 ± |
| | (>17.2) | 4.42 (7.6) | 0.64 (7.6) | 14.28 (11.7) |
| W88S | 20.7 ± | 2.49 ± | 2.39 ± | 67.09 ± |
| | 1.20 (11.9) | 0.12 (3.2) | 0.15 (2.7) | 6.76 (5.1) |
| E89G | >30 | >30 | >26.11 | 241.25 ± |
| | (>17.2) | (>39.0) | (>29.0) | 54.1 (18.5) |
| E89K | >30 | 7.21 ± | 5.21 ± | 90.2 ± |
| | (>17.2) | 0.12 (9.4) | 0.16 (5.8) | 46.24 (6.9) |
| Q161L | >30 | 8.78 ± | 6.73 ± | 114.6 ± |
| | (>17.2) | 1.58 (11.4) | 0.04 (7.5) | 14.14 (8.8) |
| Q161L/H208Y | >30 | 12.84 ± | 7.02 ± | 126.4 ± |
| | (>17.2) | 9.00 (17.7) | 1.82 (7.8) | 33.09 (9.7) |
| 4XAZT^{e} | 1.47 ± | 0.58 ± | 0.34 ± | 12.7 ± |
| | 0.63 (0.8) | 0.22 (1.2) | 0.24 (0.5) | 8.07 (0.7) |
| 4XAZTW88G | 16.37 ± | 2.46 ± | 3.31 ± | 86.2 ± |
| | 6.1 (6.2) | 0.11 (2.9) | 1.30 (4.2) | 12.16 (4.9) |
| 4XAZT/W88S | 4.09 ± | 1.54 ± | 1.51 ± | 18.44 ± |
| | 1.84 (1.6) | 0.81 (1.8) | 0.21 (1.9) | 10.40 (1.0) |
| 4XAZTE89K | 17.39 ± | 9.28 ± | 4.39 ± | 133.75 ± |
| | 14.9 (6.6) | 9.20 (11.1) | 4.59 (5.6) | 122.72 (7.6) |
| 4XAZTQ161L | 6.64 ± | 1.87 ± | 1.18 ± | 39.93 ± |
| | 2.10 (2.5) | 0.33 (2.2) | 0.61 (1.5) | 9.40 (2.3) |
| 4XAZTQ161L/ | 6.2 ± | 2.3 ± | 2.02 ± | 43.13 ± |
| H208Y | 0.85 (2.4) | 0.14 (2.7) | 0.54 (2.6) | 17.50 (2.4) |

^{a}EC₅₀ values determined measuring inhibition of p24 antigen production in MT-2 cells.
^{b}Mean ± standard deviation from at least three independent experiments.
^{c}Fold resistance relative to wild type virus
^{d}HIV-1_{LAI} encoding the indicated resistance mutations
^{e}4XAZT = D67N, K70R, T215Y, K219Q

Review of the data in Table 7 reveals that PFA resistant viruses showed similar levels of resistance to the PFA invention compounds. Virus containing the E89G mutation was least sensitive to the compounds, with fold resistance values ranging from >17.2-fold to >39.0-fold. The compounds were also tested against a panel of HIV-ILA, derived recombinant viruses containing both PFA and AZT resistance mutations. As before, the viruses demonstrated similar levels of cross-resistance to both unmodified PFA and the PFA invention compounds. In general, the presence of AZT resistance mutations decreased the level of cross-resistance to the PFA invention compounds. In the AZT resistant genetic background, mutations at codon 89 (G or K) in RT conferred the greatest degree of resistance to both unmodified PFA and PFA invention compounds with fold-resistance value.; ranging from 5.6 to 11.1.

### Example 8

Recombinant viruses containing the mutations selected by invention compounds were also evaluated for their susceptibility to AZT, as shown in Table 8.

TABLE 8
Susceptibility of Mutant Recombinant HIV-1_{LAI} to AZT

| | AZT EC₅₀^{b,c} (μM) | |
|---|---|---|
| HIV-1 Variant^{a} | resistance^{d} | Fold |
| Wild type | 0.021 ± 0.008 | |
| L214F | 0.035 ± 0.033 | 1.7 |
| S117T | 0.017 ± 0.007 | 0.8 |
| S117T/L214F | 0.022 ± 0.010 | 1.1 |
| M164I | 0.012 ± 0.004 | 0.6 |
| M164I/L214F | 0.008 ± 0.003 | 0.4 |
| W88G | 0.019 ± 0.013 | 0.9 |
| W88G/L214F | 0.015 ± 0.015 | 0.7 |
| 4XAZT^{e} | 0.290 ± 0.114 | 7.3 |
| S117T/4XAZT | 0.040 ± 0.006 | 1.4 |
| M164I/4XAZT | 0.043 ± 0.022 | 1.1 |
| W88G/4XAZT | 0.060 ± 0.018 | 1.5 |

^{a}HIV-1_{LAI} encoding the indicated resistance mutations
^{b}EC₅₀ values determined measuring inhibition of p24 antigen production in MT-2 cells.
^{c}Mean ± standard deviation from at least three independent experiments.
^{d}Fold resistance relative to wild type virus
^{e}4XAZT = D67N, K70R, T215Y, K 219Q

None of the mutations selected by the invention compounds, including L214F, reduced sensitivity to AZT. The M164I mutation was associated with an increase in AZT susceptibility (0.6-fold resistance compared to wild-type). The resistance mutations selected by the PFA invention compounds were also introduced into an AZT resistant background (D67N/K70R/T215Y/K219Q) to evaluate their effect on AZT resistance. The S117T, M164I and W88G mutations all suppressed AZT resistance from 7.3-fold to 1.4, 1.1, and 1.5-fold, respectively.

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the claims.

What is claimed is:

1. A method for treating drug-resistant human immunodeficiency virus infection in a subject in need thereof, said method comprising administering to said subject an effective amount of a lipid analog of a phosphonoformate-containing pharmaceutically active compound.

2. The method according to claim 1, wherein the lipid analog has the following structure: 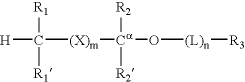
wherein:
R₁ and R₁′ are independently —H, optionally substituted —O(C₁-C₂₄)alkyl, —O(C₁-C₂₄)alkenyl, —S(C₁-C₂₄)alkyl, —S(C₁-C₂₄)alkenyl, —O(C₁-C₂₄)acyl, —S(C₁-C₂₄)acyl, wherein at least one of R₁ and R₁′ are not —H, and wherein said alkenyl has 1 to about 6 double bonds, and said acyl optionally has I to about 6 double bonds;
R₂ and R₂′ are independently —H, optionally substituted —O(C₁C₇)alkyl, —O(C₁-C₇)alkenyl, —S(C₁-C₇)alkyl, —S(C₁-C₇)alkenyl, —O(C₁C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;
R₃ is a phosphonoformate which is linked, either through its carboxyl group or its phosphonate group, to a functional group on optional linker L or to an available oxygen on C^{α}, wherein when R₃ is linked through its phosphonate group, the carboxylate group of said phosphonoformate has the following structure: 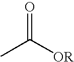
′wherein:
R_{y} is —H or alkyl, or
Na⁺, K⁺, NH₄⁺, or any other physiologically acceptable cation,
X, when present, is: 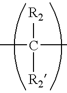
L when present is a bifunctional linking molecule of the formula —J-(CR₂)ₜ-G—, wherein t is an integer from 1 to 24, J and G are independently —O—, —S—, —C(O)O—, or —NH—, and R is —H, alkyl, or alkenyl;
m is an integer from 0 to 6; and
n is 0 or 1.

3. The method according to claim 2, wherein m is 0, 1, or 2.

4. The method according to claim 3, wherein m is 1.

5. The method according to claim 4, wherein:
R₁ is —O(C₁₈)alkyl and R₁′ is H,
R₂ is —OH, —OMethyl, or OEthyl, and R₂′ is —H,
R₂ and R₂′ on C^{α} are each —H,
R₃ is phosphonoformate, and
n is 0.

6. The method according to claim 5, wherein R₂ is —OH.

7. The method according to claim 5, wherein R₂ is —OMethyl.

8. The method according to claim 5, wherein R₂ is —OEthyl.

9. The method according to claim 1, wherein said HIV is resistant to inhibitor(s) of reverse transcriptases.

10. The method according to claim 9, wherein said inhibitors of reverse transcriptases are nucleoside analogs.

11. The method according to claim 10, wherein said nucleoside analogs are zidovudine (AZT), didanosine (ddI), zalcitabine (ddC), lamivudine (3TC), stavudine (d4T), emirivine (FTC), DAPD, DXG, tenofovir, adefovir, or abacavir.

12. The method according to claim 11, wherein said nucleoside analogs are zidovudine (AZT), didanosine (ddI), zalcitabine (ddC), or lamivudine (3TC).

13. The method according to claim 12, wherein said nucleoside analog is zidovudine (AZT).

14. The method according to claim 9, wherein said inhibitors of reverse transcriptases are non-nucleoside analogs.

15. The method according to claim 14, wherein said non-nucleoside analogs are nevirapine, delavirdine, or efavirenz.

16. The method according to claim 1, wherein said HIV is resistant to protease inhibitors.

17. The method according to claim 16, wherein said protease inhibitors are saquinavir, indinavir, ritonavir, agenerase, or DMP-450.

18. A method for the treatment of a viral infection caused by AZT-resistant strains of HIV, said method comprising administering to a subject in need thereof an effective amount of a lipid analog of a phosphonoformate-containing pharmaceutically active compound.

19. The method according to claim 18, wherein the lipid analog has the following structure: 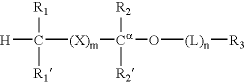
wherein:
R₁ and R₁′ are independently —H, optionally substituted —O(C₁-C₂₄)alkyl, —O(C₁-C₂₄)alkenyl, —S(C₁-C₂₄)alkyl, —S(C₁-C₂₄)alkenyl, —O(C₁-C₂₄)acyl, —S(C₁-C₂₄)acyl, wherein at least one of R₁ and R₁′ are not —H, and wherein said alkenyl has 1 to about 6 double bonds, and said acyl optionally has 1 to about 6 double bonds;
R₂ and R₂′ are independently —H, optionally substituted —O(C₁-C₇)alkyl, —O(C₁-C₇)alkenyl, —S(C₁-C₇)alkyl, —S(C₁-C₇)alkenyl, —O(C₁-C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;
R₃ is a phosphonoformate which is linked, either through its carboxyl group or its phosphonate group, to a functional group on optional linker L or to an available oxygen on C^{α}, wherein when R₃ is linked through its phosphonate group, the carboxylate group of said phosphonoformate has the following structure: 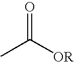
wherein:
R_{y} is —H or alkyl or
Na⁺, K⁺, NH₄⁺, or any other physiologically acceptable cation,
X, when present, is: 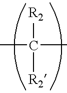
L when present is a bifunctional linking molecule of the formula —J-(CR₂)ₜ-G—, wherein t is an integer from 1 to 24, J and G are independently —O—, —S—, —C(O)O—, or —NH—, and R is —H, alkyl, or alkenyl;
m is an integer from 0 to 6; and
n is 0 or 1.

20. A method for treating a viral infection in a mammal, said method comprising administering to a subject in need thereof an effective amount of a lipid analog of a phosphonoformate-containing pharmaceutically active compound in combination with AZT.

21. The method according to claim 20, wherein said lipid analog has the following structure: 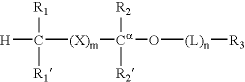
wherein:
R₁ and R₁′ are independently —H, optionally substituted —O(C₁-C₂₄)alkyl, —O(C₁-C₂₄)alkenyl, —S(C₁-C₂₄)alkyl, —S(C₁-C₂₄)alkenyl, —O(C₁-C₂₄)acyl, —S(C₁-C₂₄)acyl, wherein at least one of R₁ and R₁′ are not —H, and wherein said alkenyl has 1 to about 6 double bonds, and said acyl optionally has 1 to about 6 double bonds;
R₂ and R₂′ are independently —H, optionally substituted —O(C₁-C₇)alkyl, —O(C₁-C₇)alkenyl, —S(C₁-C₇)alkyl, —S(C₁-C₇)alkenyl, —O(C₁-C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;
R₃ is a phosphonoformate which is linked, either through its carboxyl group or its phosphonate group, to a functional group on optional linker L or to an available oxygen on C^{α}, wherein when R₃ is linked through its phosphonate group, the carboxylate group of said phosphonoformate has the following structure: 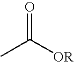
wherein:
R_{y} is —H or alkyl, or
Na⁺, K⁺, NH₄⁺, or any other physiologically acceptable cation,
X, when present, is: 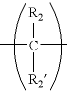
L when present is a bifunctional linking molecule of the formula
—J-(CR₂)ₜ-G—, wherein t is an integer from 1 to 24, J and G are independently —O—, —S—, —C(O)O—, or —NH—, and R is —H, alkyl, or alkenyl;
m is an integer from 0 to 6; and
n is 0 or 1.
